(19) United States
(12) Patent Application Publication
(10) Pub. No.: US 2011/0144685 A1
(43) Pub. Date: Jun. 16, 2011

(54) CRICOTHYROTOMY DEVICE

(75) Inventors: Roger Toogood, Edmonton (CA); Jason P.R. Carey, Edmonton (CA); Barry Finegan, Edmonton (CA)

(73) Assignee: GOVERNORS OF THE UNIVERSITY OF ALBERTA, Edmonton, AB (CA)

(21) Appl. No.: 12/995,739

(22) PCT Filed: Jun. 11, 2009
(86) PCT No.: PCT/CA09/00823

§ 371 Date: Feb. 11, 2011

Related U.S. Application Data
(60) Provisional application No. 61/060,675, filed on Jun. 11, 2008.

(51) Int. Cl. A61B 17/34

(52) U.S. Cl. 606/185

(57) ABSTRACT

A device for ventilating the upper airway of a trauma victim, and in particular to a device for performing a cricothyrotomy, including a inner and an outer catheter along with internal restraint that can expand inside the air way of a patient to anchor the device in place. An external restraint firmly positions the device in place.

### FIELD OF THE INVENTION

The present invention relates to a device for ventilating the airway and in particular to a device for performing a cricothyrotomy.

### BACKGROUND

It is estimated that over 3000 airway obstructed patients die each year in the United States alone due to the inability to achieve sufficient respiratory rates. Airway obstruction is often one of the conditions afflicting trauma patients; ventilating these patients provides emergency clinicians time to address the other life-threatening conditions. There are a few options to address such conditions, one of which is cricothyrotomy. Treatment may depend on the severity of the injury and no one standard treatment is routinely used (Reeder et al.). Jet ventilation using high pressure oxygen has been used successfully as an effective means to ventilate a patient in whom other means of ventilation have failed. The typical method of emergency jet ventilation is to puncture the throat at the level of the cricoid or below with a needle and insert a tube with pressurized oxygen coming in short bursts. Some known complications of the procedure include (1) directionality of the air bursts, which if not directed towards the lungs may force out the tube or cause additional trauma to the airway wall; (2) dislodgement of the tube during continuing additional trauma treatment or patient motion/struggle; (3) difficulty inserting tube after airway puncture; and (4) airway wall trauma caused by needle or the tube whipping during the air burst. A recent report from Canadian Military Medical personnel emphasized the importance of the emergency cricothyrotomy procedures and implied that a simpler method could increase the number of these procedures be performed (MacDonald et al.).

In an emergency, a cricothyrotomy can be performed by insertion of a needle into the trachea via the cricothyroid membrane, where ventilation is provided by either low pressure ventilation (at ambient atmospheric pressure, achieved by a hand pump) or with high pressure jet systems (at ˜50 psi) (Scrase et al.). The majority of currently-available devices use low pressure ventilation methods that usually involve multiple steps to perform. Consequently, there lies an opportunity in designing a new device which includes: adaptability (use with other ventilator systems such as jet ventilation or alone), quick one-step procedure, self anchoring ability in the trachea and cost effectiveness. To be competitive against the current cricothyrotomy devices, all the aforementioned design elements may contribute to the success of the new product within this narrow yet competitive market.

Therefore, there is a need in the art for a cricothyrotomy device that has the advantages of being relatively simple, effective, safe and reliable. The cricothyrotomy device should also preferably be self anchoring to enhance stability, simple to insert, and should be large enough to allow proper gas exchange.

### SUMMARY OF THE INVENTION

The present invention relates to a device for ventilating the airway of a trauma victim, and in particular to a device for performing a cricothyrotomy.

In one aspect, the invention comprises a device for performing a cricothyrotomy on a patient, the device comprising;
(a) an inner catheter having an inner bore and an outer surface, and having a distal end and a proximal end, the distal end ending in a directional nozzle;
(b) an internal restraint mounted on the outer surface of the inner catheter adjacent the directional nozzle, the internal restraint having a relaxed shape allowing entry into the patient, and an activated shape restraining the device within the patient;
(c) an outer catheter having an inner bore and an outer surface, the outer catheter slidably disposed on the inner catheter and movable between a first position and a second position whereby the outer catheter activates the internal restraint; and
(d) an external restraint mounted on the outer surface of the outer catheter.

### BRIEF DESCRIPTION OF THE DRAWINGS

In the drawings, like elements are assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention. The drawings are briefly described as follows:

FIG. 1 is a diagrammatic depiction of one embodiment of the assembled device.

FIG. 2 is a diagrammatic depiction of the inner catheter of the embodiment shown in FIG. 1.

FIG. 3 is a diagrammatic depiction of the inner restraint in a relaxed shape.

FIG. 4 shows the inner restraint in its radially expanded activated shape.

FIG. 5 shows the outer catheter of the embodiment shown in FIG. 1.

FIG. 6 shows an exploded view of the distal end of the device.

FIG. 7 shows one embodiment of an external restraint.

FIG. 8 shows an external restraint clip.

FIG. 9 shows one embodiment of a one-way valve.

### DETAILED DESCRIPTION OF PREFERRED EMBODIMENTS

The present invention relates to a device for performing a cricothyrotomy. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

A “Luer fitting” is a standardized system of small-scale fluid fittings used for making leak-free connections between a male-taper fitting and its mating female part on medical and laboratory instruments, including hypodermic syringe tips and needles or stopcocks and needles. A “Luer lock” means a type of the fitting connector with locking mechanism, used extensively for medical and laboratory applications. “Luer-Lok” and “Luer-Slip” are registered trademarks of Becton Dickinson.

“Catheter” as used herein means a hollow flexible tube for insertion into a body cavity or duct.

The device (10) comprises a cricothyrotomy device that can be easily and quickly affixed in place once inserted into the airway of a patient. The underlying premise of the device is the ability to insert a catheter through the cricothyroid tissue using a guide needle, and, following removal or retraction of the guide needle, to then expand an internal restraint inside the airway of the patient to anchor the device in place. An external restraint system further ensures proper fixation of the device. To deliver the required inflow, an air supply is connected to the device to ventilate the victim, which is preferably a high pressure oxygen supply.

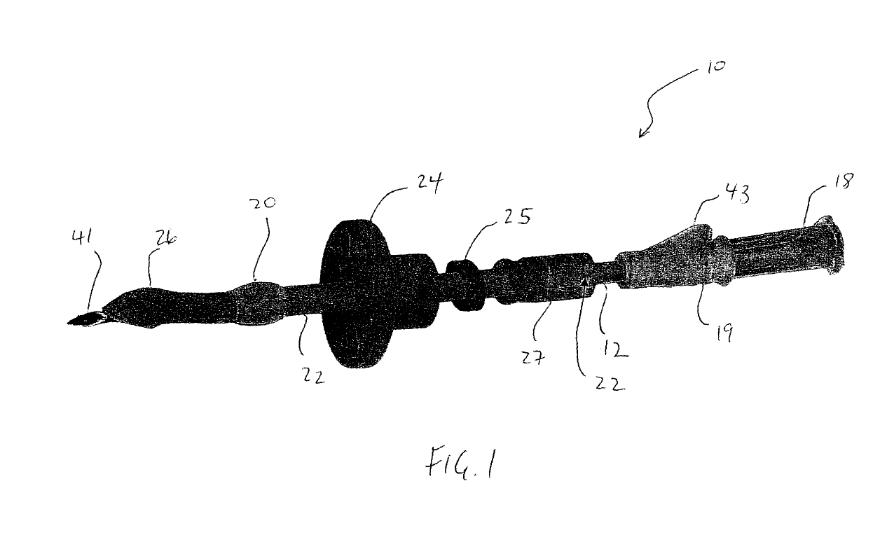

One embodiment of the cricothyrotomy device (10) is shown in FIG. 1. The device (10) has an inner catheter (12) fitted with a directional nozzle (26) through which air or oxygen is directed into the patient, an internal restraint (20), an external restraint (24), an outer catheter (22), a fitting (18) for attachment to an air or oxygen supply and an external restraint lock (25).

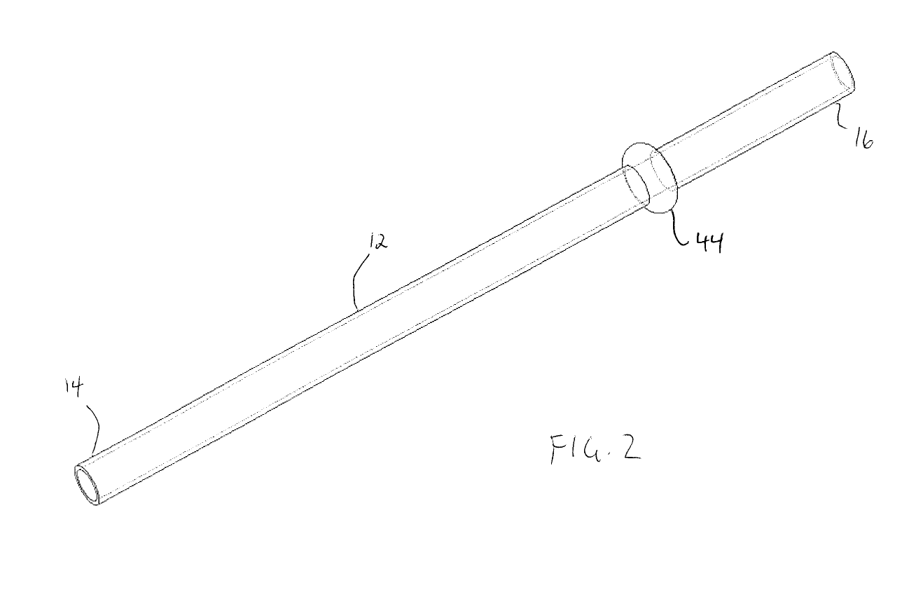

As shown in FIG. 2, the inner catheter (12), which is preferably moderately flexible, has an inner bore and an outer surface. The directional nozzle (26) is fitted onto the inner catheter at its distal end (14). The fitting for attachment to an air or oxygen supply (18) at the proximal end (16) of the inner catheter (12) may, in one embodiment, comprise a female Luer fitting. The nozzle is directional because it is curved in a downward direction, which directs the air or oxygen downward in the patient's trachea, towards the lungs. A high pressure gas jet directed at the tracheal wall opposite the device insertion point may cause further injury to the patient.

The guide or insertion needle (41) passes through the inner catheter and is exposed out of the nozzle. Because the insertion needle (41) is preferably straight, the nozzle must retain some resilient flexibility to accommodate the needle, and then to regain its directional shape when the needle is withdrawn. In an alternative embodiment, the nozzle itself may be sharpened or pointed to provide insertion means into the patient, forgoing the need for a needle.

The directional nozzle (26) may be comprised of a resiliently flexible material. In one embodiment, strategically placed reinforcement fibres may be provided in the nozzle to bias the tip in its curved shape and in particular to prevent straightening of the curved tip during the pressurized air pulse.

In a preferred embodiment, the catheter over insertion needle design provides for rapid insertion. The device (10) is designed to comprise a current upper airway puncture 12 gauge syringe system (not shown), and is therefore relatively simple to insert. As shown in FIG. 1, at the time of insertion when the inner catheter (12) is mounted on the insertion needle (41) of the syringe system, the directional nozzle (26) flexes to accommodate the insertion needle (41). Following insertion and removal of the insertion needle (41), the nozzle (26) will revert back to its curved shape and will curve back such that it points towards the lungs.

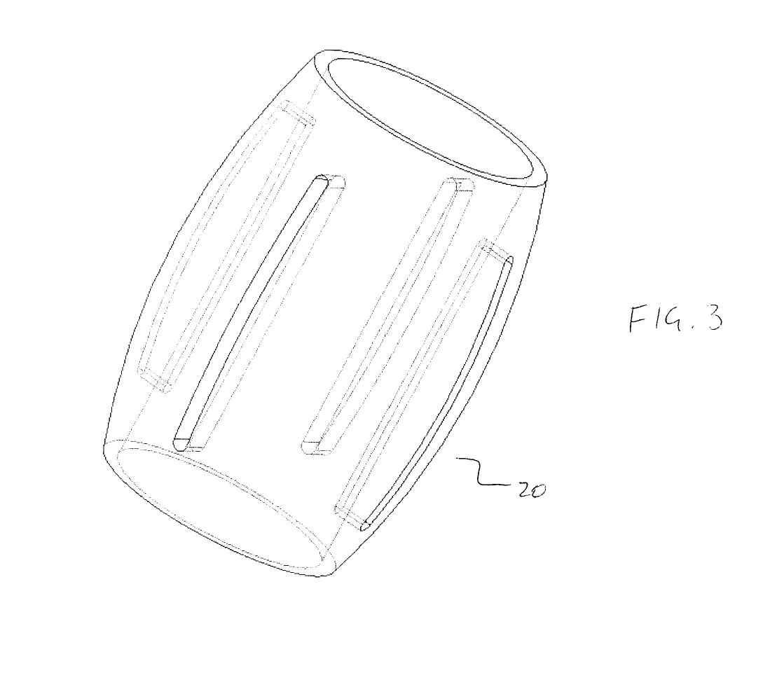

The proximal end (16) of the inner catheter comprises a fitting for attachment to an air or oxygen supply (18). Any suitable attachment fitting may be used that will facilitate a gas tight seal and which is suitable for attachment to a gas hose. The attachment fitting (18) preferably has a valve or other means to control directional flow of the air flow. Preferred attachment means is a female Luer fitting (18), which accepts a male Luer lock coupler attached to a pressurized air hose. In one embodiment, as seen in FIGS. 1 and 3, the attachment fitting (18) may include a member (19) has at least one projection (43) which acts as a directional guide or indicator. The projection (43) will indicate to the practitioner the orientation of the directional nozzle (26), so that the nozzle is correctly oriented in the patient before a high pressure gas jet is applied. This directional aspect of the gas delivery is important for a cricothyrotomy device because if the air is not delivered to the lungs, it may force the cricothyrotomy device out of the patient or it may cause trauma to the airway wall which can result in blood filling the patient's lungs. The directional indicator (43) of the present device in conjunction with the directional nozzle (26) mitigate the risk of this complication.

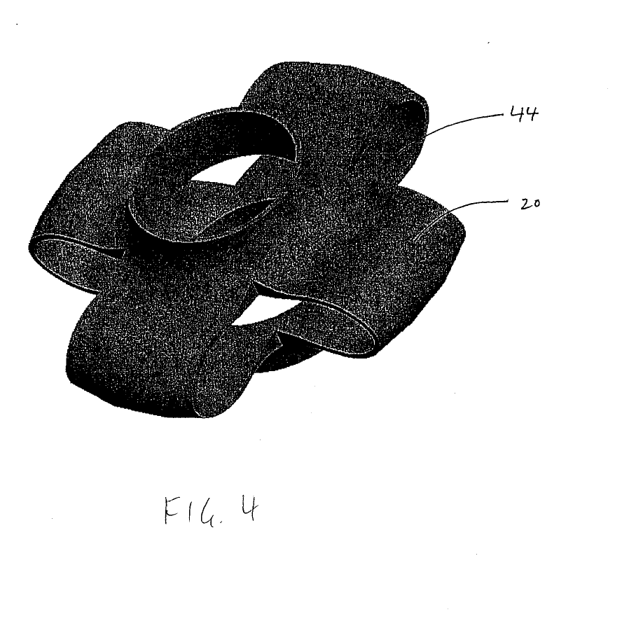

Mounted on the exterior surface of the inner catheter (12) adjacent to the nozzle (26) is an internal restraint (20), which functions to anchor the device within the trachea. The internal restraint (20) may take a relaxed shape which facilitates entry into the patient through the airway wall, and an activated shape which resists reverse passage through the airway wall opening. The activated shape may comprise a radially expanded shape. In one embodiment, the internal restraint comprises an elastic tubular sleeve, which is preferably made from bio-compatible material that can withstand large deformations and which is resiliently deformable. In one embodiment, the sleeve has at least two slits in its walls, and preferably three or four slits. In an alternative embodiment, the sleeve may define a plurality of openings, such as a mesh or web structure. The exact configuration of the sleeve which permits radial expansion is not an essential element of the invention, and one skilled in the art may conceive of alternative configurations. When the sleeve is in an uncompressed state, it assumes the relaxed tubular shape conforming to the inner catheter. However, when axially compressed, the sleeve deforms and expands radially outward assuming an activated shape. If the inner restraint is tubular with 4 slits, the activated shape will comprise cruciform arms (44) as shown in FIG. 4. The extended arms (44) therefore anchor the device when held against the inner wall of the airway.

During insertion of the device (10), the internal restraint (20) is in its uncompressed relaxed shape as shown in FIG. 1 or 3. Once inserted, the internal restraint is axially compressed by contact by the outer catheter (22) as described below.

A concern regarding the internal restraint (20) is permanent deformation which would complicate extraction of the device. Once expanded and left for the short term (1-2 hrs for example) in the victim's airway, the expanding restraint could potentially permanently deform aided by the humid and body temperature environment. Therefore, the chosen material for the inner restraint must be biologically compatible and allow very large and (preferably) reversible deformations without failure or fracture. In one embodiment, the inner restraint (20) may be a disposable element, and therefore design consideration allowing repeated reuse may not be required.

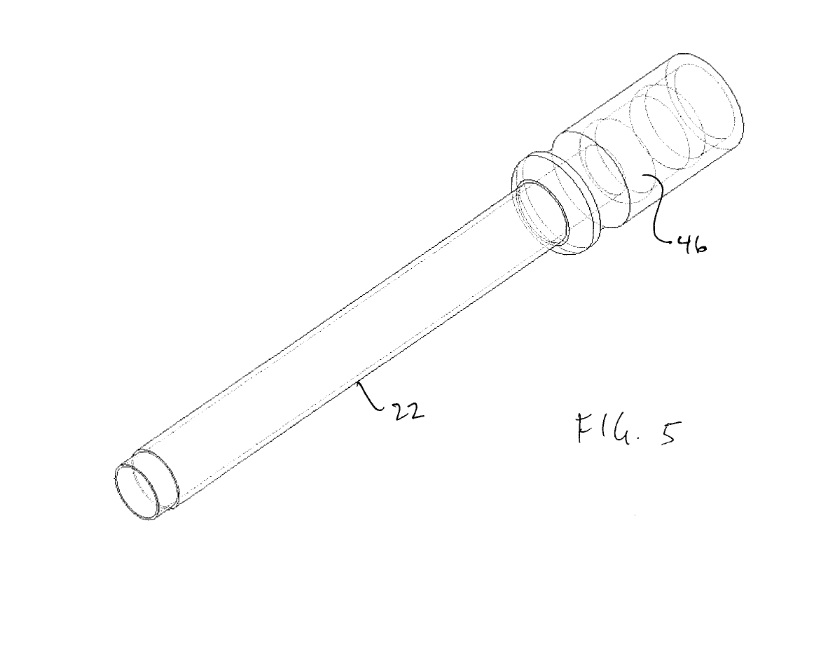

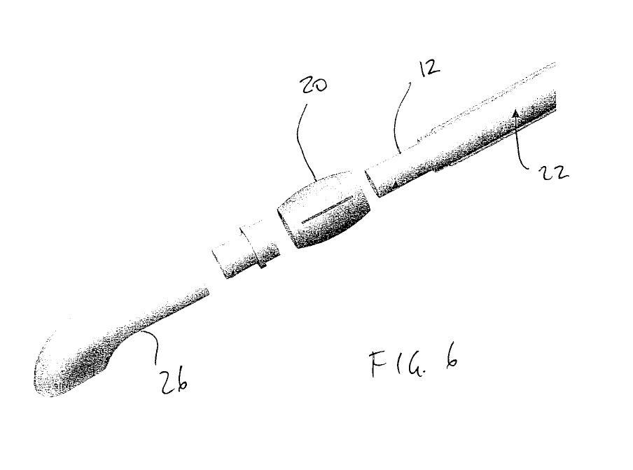

As shown in FIGS. 5 and 6, the outer catheter (22) has an inner bore and an outer surface. The outer catheter (22) is slidably disposed on the outer surface of the inner catheter (12) as shown in FIG. 1 with the inner catheter (12) running through the inner bore of the outer catheter (22). As shown in FIG. 1, during insertion of the device (10), the outer catheter (22) is in a first position in which it does not compress the inner restraint (20) mounted on the distal end of the inner catheter (14). However, following insertion, the outer catheter may be moved distally on the inner catheter (14) by grasping the inner catheter (12) and sliding the outer catheter (22) towards the distal end of the inner catheter (14). The impingement of the outer catheter (22) on the internal restraint (20) causes the internal restraint (20) to deform and assume its activated shape. Retraction of the outer catheter (22) from its second position towards the proximal end of the inner catheter (16) into its first position will disengage the outer catheter (22) from the internal restraint (20) and it will return to its uncompressed relaxed state. It can be understood that prior to removal of the device from the patient, the outer catheter (22) will be disengaged from the internal restraint (20) to prevent physical trauma to the airway and the puncture wound upon extraction.

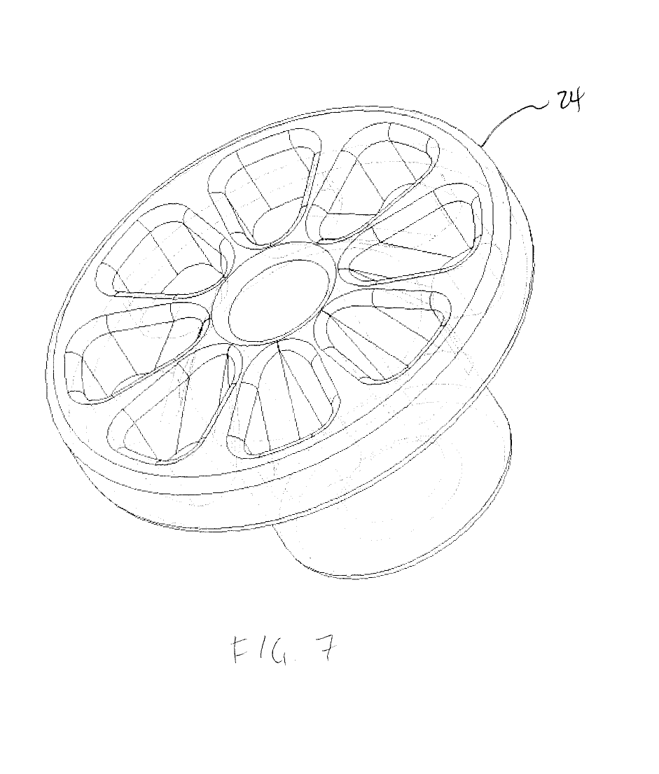

An external restraint (24) is slidably mounted on the outer surface of the outer catheter. As shown in FIGS. 1 and 7, in one embodiment, the external restraint (24) is a disk which slides up and down the outer surface of the outer catheter (22). The external restraint may take any shape which will not affect its functionality. In one embodiment, the restraint may be somewhat flexible around its periphery, but stiffer where it connects to the outer catheter. The external restraint may be perforated and may be textured to prevent slipping. Following insertion of the device and upon activation of the internal restraint (20), the external restraint is moved distally along the outer catheter (14) into a position whereby it engages the patient's neck. As will be understood by one skilled in the art, the patient's airway wall will be firmly held between the inner and external restraints.

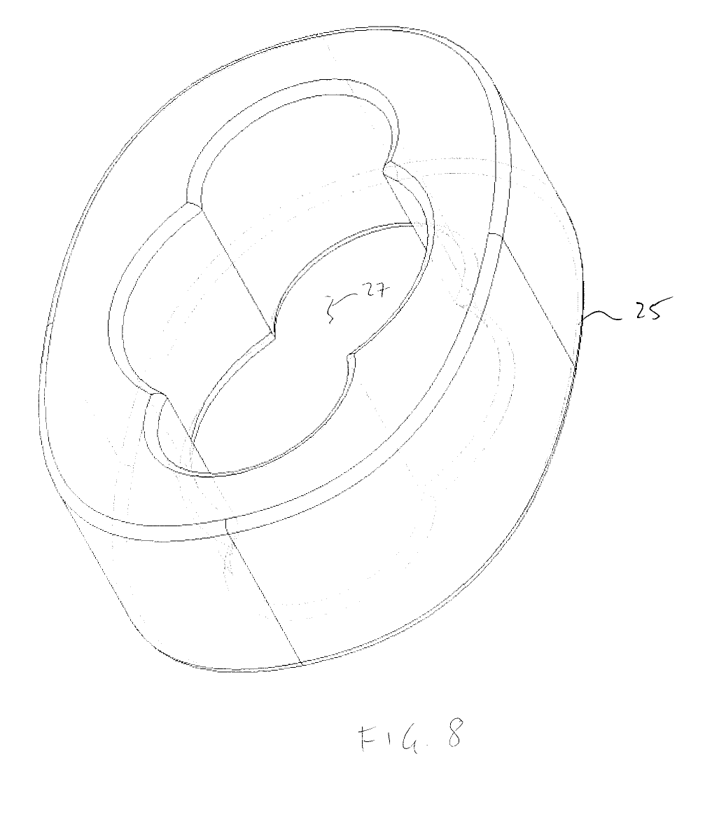

In one embodiment, the external restraint (24) may slide along the outer catheter with relative ease, to facilitate its use. However, the external restraint will easily move out of place. Therefore, in one embodiment, a restraint clip (25) may be provided to prevent proximal movement of the external restraint once it has been positioned. The restraint clip may be designed as a resiliently deformable clip, which is moveable along the outer catheter when deformed, but snugly grips the outer catheter when relaxed. One embodiment of such a restraint clip is shown in FIG. 8. The restraint clip (25) has an oval shape, with an oval opening (27). When pinched from the elongate ends, the height of the opening (27) expands allowing easy movement along the outer catheter. When relaxed, the oval opening tightly grips the outer catheter. It can be understood that any other suitable securing clips or configurations as might be selected by one skilled in the art may be used to lock the external restraint on the outer catheter.

In one embodiment, the outer catheter (22) may comprise a series of graduated markings (27) that may comprise notches, rings or markings on the outer surface of the outer catheter. The graduated markings (27) show how deep the device has been inserted into the patient, thus aiding the user in inserting the device to an appropriate depth. In one embodiment, the graduated markings may cooperate with one or both of the external restraint or the restraint clip to assist in releasably locking the external restraint in position.

The capability of the device (10) to allow proper airflow has been evaluated. It has been suggested that a minimum pressure of 400 kPa to successfully inflate the lungs, with a flow rate of 800 mL/sec through a 4 mm cannula. A 50 psi inlet source is preferable, with a minimum 16 gauge for inhalation. A 12 gauge catheter is preferable for exhalation and hence a 12 gauge catheter is used in one embodiment of the present invention. Flow requirements and restriction may be calculated to evaluate the adequacy of the device geometry. In one embodiment, the device comprises a 50 mm long catheter, a flow rate of 50 L/min and an inlet pressure of 400 kPa, which results in a 1.564 psi pressure drop.

Exemplary use of the device (10) to ventilate a patient will now be described; however, the described method of use is not intended to limit the claimed invention. The device (10) is mounted on the insertion needle (41) of an upper airway puncture 12 gauge syringe system (40). The outer catheter (22) is positioned proximally on the inner catheter. The needle (41) is used to puncture the throat at the level of the cricoid and the insertion needle (41) and the directional nozzle of the inner catheter (12) are inserted into the patient's airway. The graduated markings (27) on the outer catheter (22) assist the user to gauge the correct positioning of the tip of the insertion needle in the airway. As well, retraction of the syringe may be performed to confirm if airflow is possible through the device. The device is rotated so that the direction indicator (43) is properly aligned thereby ensuring that the directional nozzle (26) is pointing towards the patient's lungs. Once correct positioning is determined, the syringe system (40) and insertion needle (41) are extracted. The inner catheter (12) is grasped and the outer catheter (22) is pushed in a direction towards the distal end of the inner catheter (14) moving the outer catheter (22) to activate the internal restraint (20). In one embodiment, the range of motion of the outer catheter relative to the inner catheter to precisely control the expansion of the inner restraint is determined by a circumferential ridge (44) on the outer surface of the inner catheter and a matching circumferential groove (46) on the inner surface of the outer catheter. The ridge (44) is constrained by the length of the groove (46).

The internal restraint (20) is compressed between the outer catheter (22) and the nozzle, and it deforms outwardly. The device is then moved proximally until the surface of the expanded inner restraint contacts the wall of the patient's airway. The external restraint (24) is lowered into its second position whereby it engages the patient's neck and locked into place using the restraint clip. The device is now secured on the patient's neck. The device is then attached to a high pressure oxygen hose by means of the Luer fitting (18) at the proximal end of the inner catheter (16). It is possible to fit a manually ventilated airbag to the device, however, the source of pressurized air is not an essential element of the invention.

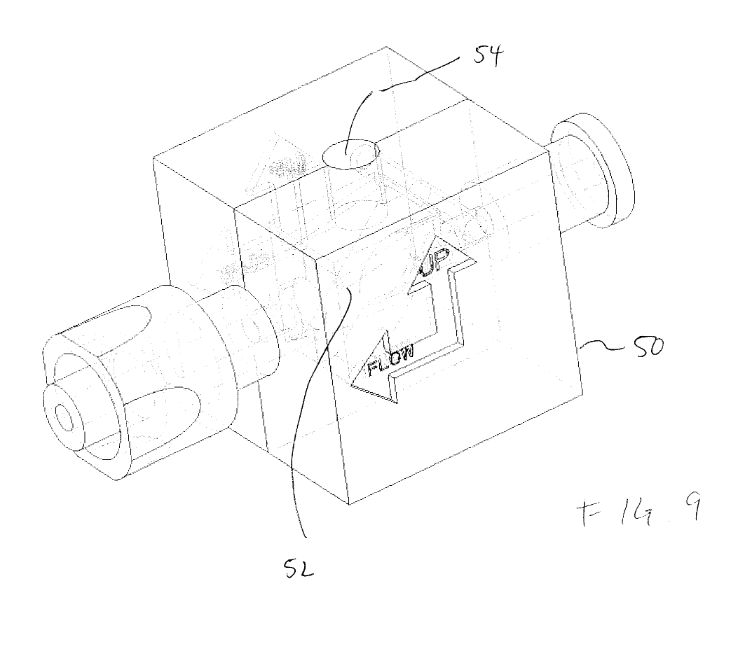

Ventilation using, for example, short blasts of high pressure oxygen may then be commenced. In one embodiment, a one-way valve (50) as shown in FIG. 9 may be fitted to the device. A flapper valve (52) permits air pressure to flow to the inner catheter while blocking the vent (54). As the patient exhales, the flapper valve (52) blocks the air pressure source and allows exhaled air to escape the vent (54). Other valves such as a ball valve may be suitable and selected by one skilled in the art.

To extract the device (10), the restraint clip is moved allowing the external restraint to be loosened, and the inner catheter (12) is moved distally, thereby deactivating the internal restraint (20). The device (10) is pulled out of the patient's airway.

The device may be constructed from any suitable biocompatible materials as are commonly used in the art. A series of materials have been evaluated by the inventors to manufacture the different components. Any suitable polymers may be used, but preferably the polymers will be biocompatible, can be injection moulded and can be sterilized by autoclave prior to packaging. Pursil A1 20, Carbosil 55D, Pellathane 2363-80A and Elasthane 80A series of polymers (Polymer Technology Group, Berkely, Calif., USA) were evaluated and used in the FEA. Those skilled in the art will be able to choose suitable and preferred materials with routine testing and analysis.

As will be apparent to those skilled in the art, various modifications, adaptations and variations of the foregoing specific disclosure can be made without departing from the scope of the invention claimed herein.

The following references are cited in the application at the relevant portion of the application. Each of these references is incorporated herein by reference, where permitted.
Reeder, T. J., Brown, C. K., and Norris, D. L, Managing the difficult airway: a survey of residency directors and a call for change, The Journal of Emergency Medicine, 28(4): 473-478, 2005.
MacDonald, J. C., Tien, H. C. N., Emergency battlefield cricothyrotomy, Canadian Medical Association Journal, 178(9): 1133-1135, Apr. 22, 2008.
Scrase, and Woollard. Needle vs surgical cricothyrotomy: a short cut to effective ventilation. Anaesthesia, 61:962-974, 2006.

1. A device for performing a cricothyrotomy on a patient, the device comprising:
(a) an inner catheter having an inner bore and an outer surface, and having a distal end and a proximal end, the distal end ending in a directional nozzle;
(b) an internal restraint mounted on the outer surface of the inner catheter adjacent the directional nozzle, the internal restraint having a relaxed shape allowing entry into the patient, and an activated shape restraining the device within the patient;
(c) an outer catheter having an inner bore and an outer surface, the outer catheter slidably disposed on the inner catheter and movable between a first position and a second position whereby the outer catheter activates the internal restraint; and
(d) an external restraint mounted on the outer surface of the outer catheter.

2. The device of claim 1 further comprising a needle disposed within the inner catheter wherein a distal end of the needle is exposed from the nozzle.

3. The device of claim 2 further comprising a syringe adapted to attach to a proximal end of the needle.

4. The device of claim 1 further comprising an external restraint clip slidably engaging the outer catheter, for locking the external restraint into position on the outer catheter.

5. The device of claim 1 wherein the outer catheter comprises depth indication markings along the length of the outer catheter.

6. The device of claim 1 further comprising a directional indicator which shows the orientation of the directional nozzle when inserted into a patient.

7. The device of claim 1 wherein the internal restraint comprises a resiliently elastic sleeve.

8. The device of claim 7 wherein the internal restraint comprises a resiliently elastic sleeve having 2, 3 or 4 slits.

9. The device of claim 1 wherein the external restraint comprises a disc.

10. The device of claim 4 wherein the restraint clip comprises an elastic member having a central opening, wherein one dimension of the opening may be enlarged by deforming the elastic member.

11. The device of claim 1 wherein the directional nozzle directs air in a direction about 90° from the inner catheter.

12. The device of claim 3 wherein the directional nozzle is sufficiently flexible to allow the needle to be exposed from the end of the nozzle and sufficiently rigid to maintain its directionality when pressurized air is directed through it.

13. The device of claim 12 wherein the directional nozzle is fibre reinforced.

14. The device of claim 1 further comprising a movement range stop disposed between the inner catheter and outer catheter which limits the range of movement of the outer catheter relative to the inner catheter.

15. The device of claim 14 wherein the movement range stop comprises a projection disposed on the outer surface of the inner catheter which travels within a groove defined in the inner bore of the outer catheter.

16. The device of claim 1 further comprising a one-way valve defining an exhalation vent, permitting gas flow distally through the device from an air pressure source.

17. The device of claim 16 wherein the one-way valve comprises a flapper valve.
